(19) United States
(12) Patent Application Publication
(10) Pub. No.: US 2026/0338279 A1
(43) Pub. Date: Oct. 29, 2026

(54) Rotary Valve

(71) Applicant: EMD Millipore Corporation, Burlington, MA (US)

(72) Inventors: Nathan Landry, Burlington, MA (US); James Ormond, Belmont, MA (US); Jeffrey Knox, Peabody, MA (US); Kathy Youngbear, Cambridge, MA (US); Christina Carbrello, Bedford, MA (US); Benjamin Cacace, Tewksbury, MA (US); Joseph Geringer, Merrimack, NH (US)

(21) Appl. No.: 19/120,607

(22) PCT Filed: Oct. 13, 2023
(86) PCT No.: PCT/US2023/076763

§ 371 Date: Apr. 11, 2025

Related U.S. Application Data
(60) Provisional application No. 63/379,336, filed on Oct. 13, 2022.

(51) Int. Cl. F16K 3/06; B01D 63/08; F16K 27/04; F16K 31/60

(52) U.S. Cl. CPC F16K 3/06; B01D 63/082; F16K 27/045; F16K 31/602; B01D 2313/18

(57) ABSTRACT

A rotary genderless valve (500) having a body (502) having a body hole (524); a body cover (510) for attaching to the body, the body cover comprising a cover hole (522) and optionally comprising alignment holes (514); a handle (504), wherein the handle moves from a closed position and an open position actuated by a rotary motion; and a rotary slide (528) attached to the handle capable of partial rotation, wherein the rotary slide has an elastomeric slide which comprises a rotary slide hole (526); and, optionally, a second genderless valve having a second rotary slide and rotary slide hole, wherein the rotary slide hole and the second rotary slide hole are in fluid communication with one another.

The present application claims the benefit of priority to U.S. Provisional Application No. 63/379,336, dated Oct. 13, 2022 which is incorporated by reference in its entirety.

### FIELD OF THE DISCLOSURE

This disclosure relates to filtration. More particularly, embodiments described herein relate to connectors and valves for filtration cartridges.

### BACKGROUND OF THE DISCLOSURE

Many downstream filtration products, like the Viresolve® Pro Magnus Device product family, sold by EMD Millipore Corporation, Burlington, MA, USA, have cassette style designs, allowing multiple devices to be assembled in one holder. Stackable designs enable users to size an assembly to fit their needs, i.e., eliminating waste, while keeping smaller footprints and low working volumes. However, during the assembly of a plurality of devices into a holder, the devices are exposed to the ambient conditions of the room, which may contaminate the devices with microorganisms. To combat contamination, customers will often clean the devices after assembly with a cleaning solution, assemble the devices in a clean environment, or rely on sterile filters downstream to remove any contaminants. Recently, industries are shifting toward providing devices and products that are delivered pre-sterilized. Furthermore, these devices often include aseptic connections to further reduce the contamination risk to devices and/or processes. The integration of pre-sterilized and aseptically connected devices enables users to assemble the devices in a non-controlled environment, while keeping the sterility of the flow path intact. Current products on the market attach external sterile connectors to the devices and provide external manifolds to connect multiple devices together. However, this approach creates many issues for users including, though not limited to, increased cost, higher hold up volumes, poor usability, reduced capacity, reduced sustainability, and increased risk of connection issues, i.e., leaking and loss of sterility.

Small filters have been hand-assembled for parallel flow with supporting plates and associated apparatus, then tested, and, if necessary, sterilized, often at the user's site at considerable cost, inconvenience, and risk. The operations must be repeated if the hand assembly fails the necessary tests. The mechanical parts of larger more complex filtration systems are generally cleaned and re-used with only the filters being replaced. One previous single use assembly provided has also been mechanically secured with relatively non-movable parts, i.e., fixed and/or rigid piping.

Individual membrane filters of large area have been supported flat or cylindrically. Alternatively, pleated membrane filters have been disposed within compact housings. Holders for flat membranes are large, for a given filter area, and are usually not disposable, require disassembly, cleaning, re-assembly, and integrity testing with each change of filter. Pleating of fragile membranes creates stress concentrations at the folds, permits flexing of the fragile membranes in use, and normally employs interleaving flow screens on one or both of upstream and downstream sides. Also, because of concerns for possible failures at the folds, seams, or ends, a separate flat final filter is sometimes used in series with pleated cartridges for added assurance in critical applications, for example, in sterilizing pharmaceuticals and intravenous fluids, which adds complexity and cost. Membrane filters comprising various polymeric materials are known and are thin, porous structures having porosities between approximately 50-80% by volume. These membranes are relatively fragile and are used with various mechanical supports or reinforcements. Flow rates of liquids through such membranes per unit of area are a function of pore size and structure. To obtain high flow rates through filters with fine pores, e.g., below approximately one micron, relatively large filter areas are needed. To accommodate these needs, i.e., sterile, large individual filters or a larger number of smaller individual filters are used in parallel. For use within some applications, e.g., life sciences/pharma, the membranes and their supporting apparatus must be free of leaks.

Biological fluids, which may have bacteria, micro-organisms, etc., are processed in available filter cartridges that undergo significant pressure drop, limiting the volume of a biological fluid that can be processed through the cartridge. The degree of pressure drop is closely related to the membrane resistance, which is owing to the porosity and pore size, i.e., the pore structure, and the flow path length of the fluid within the cartridge, i.e., the longer the flow path length, the greater the pressure drop.

Past prior art attempts to solve these issues include using a filtration cartridge formed from a plurality of stacked filtration modules and having a separate exterior housing. Cartridges are disfavored because of large hold up volumes, which result in significant sample loss. Another technology included a filtration cartridge having a feed inlet and a permeate outlet positioned at a central portion of the cartridge. This cartridge requires a fluid deflection plate to direct incoming fluid feed from a central portion of the cartridge to a peripheral portion of the cartridge. Deflection plates are disfavored because these include non-working elements within the cartridge.

Integrity testing is required of filters. Integrity testing sometimes employs a binary gas system for determining the presence of defects within membranes or filter devices. To perform this type of testing, binary gas must be flowed across the membrane in tangential flow filtration (TFF) as opposed to a normal flow filtration mode (NFF), also known as, dead-end filtration. Integrity testing of this type is described in U.S. Pat. No. 7,594,425, filed Oct. 10, 2006, and is incorporated herein in its entirety. Accordingly, when it is desired to effect NFF filtration within a filtration cartridge and to effect the integrity test, the filtration cartridge may be capable of being operated in both TFF and NFF modes.

It would be desirable to provide a filtration cartridge having a single feed inlet and a single permeate outlet for simplicity. In addition, it would be desirable to provide a cartridge that can be operated in both TFF and NFF modes. Furthermore, it would be desirable to provide a filtration cartridge with a minimum of non-working elements to reduce cost. Further still, inserts providing an effective way to connect and maintain sterility between filter cartridges without manifolds would be an advance in the art. Moreover, providing a cartridge wherein the fluid being processed experiences a low pressure drop within the cartridge as compared to presently available cartridges represents an advance in the art.

A rotary genderless valve having a body having a body hole; a body cover for attaching to the body, the body cover comprising a cover hole and optionally comprising alignment holes; a handle, wherein the handle comprises a closed side and an open side actuated by a rotary motion; and a rotary slide attached to the handle capable of partial rotation, wherein the elastomeric slide comprises a rotary slide hole; and, optionally, a second genderless valve having a second rotary slide and rotary slide hole, wherein the rotary slide hole and the second rotary slide hole are in fluid communication with one another.

### SUMMARY OF THE DISCLOSURE

Connectors for use in bioprocessing are disclosed. Filtration is at least one bioprocessing operation that can be performed using the embodiments of the disclosure. Some embodiments of connectors, also called valves, described herein, substantially as shown in and/or described in connection with at least one of the figures, as set forth more completely in the claims, are disclosed. Embodiments of connectors described within the disclosure eliminate the drawbacks summarized above by creating, in some embodiments, an integrated gamma irradiatable sterile connection, or sterile barrier, into the ports or endcaps of the stacked cassette devices. Any of the connectors, plugs or inserts described herein can be sterilized via gamma, x-ray, and/or sterilization by ionizing radiation. These connectors, or barriers, permit users to assemble the devices aseptically, while eliminating the need for external manifolds, third party connectors, and accessories.

The connectors described in this disclosure can be integrated, for example, into end caps or ports of filtration devices, cartridges, and/or holders. Linearly-actuated and rotary-actuated genderless valves are also disclosed. The connectors and genderless valves described herein are not exclusive to viral clearance cassettes and can also be used in other cassette style devices within the filtration industry, such as membrane chromatography devices and clarification devices, as well as other liquid management industries. Various benefits, aspects, novel and inventive features of the disclosure, as well as details of exemplary embodiments thereof, will be more fully understood from the following description and drawings.

### BRIEF DESCRIPTION OF THE DRAWINGS

FIG. 1 depicts a front view of a connector, according to some embodiments of the disclosure;

FIG. 2 is a cross-section view taken along line 2-2 of a connector of FIG. 1, according to some embodiments of the disclosure;

FIG. 3 is an exploded view of a connector of FIGS. 1-2, according to some embodiments of the disclosure;

FIG. 4 is a front perspective view of a filtration cassette that can be connected via the connector of FIGS. 1-3, according to some embodiments of the disclosure;

FIG. 5 is a cross-section view taken along line 5-5 of one cassette, according to some embodiments of the disclosure;

FIG. 6 depicts the cross-section views of two cassettes 200, wherein the two cassettes 200 are stacked and connected via connectors 100, according to some embodiments of the disclosure;

FIG. 7 is a front perspective view of a valve, according to some embodiments of the disclosure;

FIG. 8 is a front perspective view of the valve of FIGS. 7C-7D, according to some embodiments of the disclosure;

FIG. 9 is a cross-section view taken along line 9-9 of the valve 300 of FIG. 7A and further including a complementary valve 300 in closed positions, according to some embodiments of the disclosure;

FIG. 10 is a cross-section view taken along line 10-10 of a valve 300 of FIG. 7D and further including a complementary valve 300 in open positions, according to some embodiments of the disclosure;

FIG. 11 depicts a close view of the cross-sections of FIGS. 9-10, according to embodiments of the disclosure;

FIG. 12 depicts FIGS. 12A-12B showing two complementary rotary aseptic valves in closed positions in an exploded view, according to some embodiments of the disclosure;

FIG. 13 depicts FIGS. 12C-12D showing two complementary rotary valves in open positions in an exploded view, according to some embodiments of the disclosure;

FIG. 14 depicts a cross-section of FIGS. 12A-12B showing two complementary rotary aseptic valves in closed positions in an assembled view, according to some embodiments of the disclosure;

FIG. 15 depicts a cross-section of FIGS. 12C-12D showing two complementary rotary valves in open positions in an assembled view, according to some embodiments of the disclosure;

FIG. 16 depicts an exploded view of the two rotary valves of FIGS. 12-15, according to some embodiments of the disclosure;

FIG. 17 depicts a rotary valve having an air gap, according to some embodiments of the disclosure;

FIG. 18 depicts an exploded view of two rotary valves, wherein the rotary valve is as shown in FIG. 17, according to some embodiments of the disclosure;

FIG. 19 depicts a partially exploded view of a genderless expandable valve, according to some embodiments of the disclosure;

FIG. 20 depicts two perspective exploded views of the genderless expandable valve of FIG. 19, according to some embodiments of the disclosure;

FIG. 21 depicts sliders for use within a genderless expandable valve, according to some embodiments of the disclosure;

FIG. 22 depicts a perspective view of the genderless expandable valve of FIGS. 19-21 in open and closed positions, according to some embodiments of the disclosure;

FIG. 23 depicts a sectional view of the genderless expandable valve of FIGS. 19-22, according to some embodiments of the disclosure; and FIG. 24 depicts a partially exploded view of a second genderless expandable valve, according to some embodiments of the disclosure.

### DETAILED DESCRIPTION

Although specific terms are used in the following description for the sake of clarity, these terms are intended to refer only to the particular structure of the embodiments selected for illustration in the drawings. In the drawings and the following description below, it is to be understood that like numeric designations refer to components of like structure and/or function.

The singular forms “a,” “an,” and “the” include plural referents unless the context clearly dictates otherwise.

As used in the specification, various devices and parts may be described as “comprising” other components. The terms “comprise(s),” “include(s),” “having,”“is,” “has,” “can,” “contain(s),” and variants thereof, as used herein, are intended to be open-ended transitional phrases, terms, or words that do not preclude the possibility of additional components.

All ranges disclosed herein are inclusive of the recited endpoint and independently combinable (for example, the range of “from 2% to 10%” is inclusive of the endpoints, 2% and 10%, and all intermediate values).

As used herein, approximating language may be applied to modify any quantitative representation that may vary without resulting in a change in the basic function to which it is related. Accordingly, a value modified by a term or terms, such as “about,” “approximately,” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.”

It should be noted that some terms used herein are relative terms. For example, the terms “upper” and “lower” are relative to each other in location, i.e., an upper component is located at a higher elevation than a lower component and is not to be construed as requiring a particular orientation or location of the structure. As a further example, the terms “interior,” “exterior,” “inward,” and “outward” are relative to a center and should not be construed as requiring a particular orientation or location of the structure.

The terms “top” and “bottom” are relative to an absolute reference, i.e., the surface of the earth. Put another way, a top location is always located at a higher elevation than a bottom location, toward the surface of the earth. The term “genderless” within this disclosure indicates that a flow path of liquid can be made without any flow channel projecting into another part of another flow channel. A “flow channel” or flow path is any passage capable of allowing fluid flow therethrough.

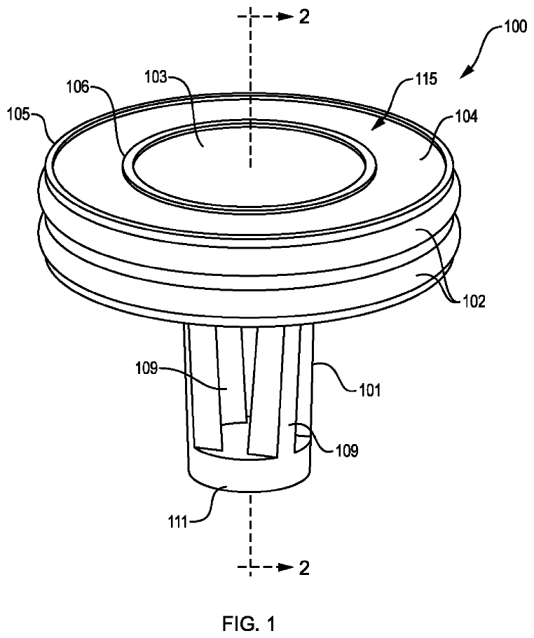

Embodiment One: Bi-Modal Membrane Insert. The bi-modal membrane insert outlines a method of preventing the contamination of devices, e.g., stackable devices, using a sterile barrier. The sterile barrier, e.g., a membrane insert, is inserted into ports of the stackable device and secured in place by means of, e.g., welding and/or, i.e., elastomeric seals, e.g., o-rings, or other means capable of creating a hermetic seal between a frame and a stackable device, e.g., a cassette and/or between two adjacent cassettes. In some embodiments, the membrane is welded to the device. The sterile barrier/membrane insert may be placed within the device throughout shipping, storage, and use. In addition, the membrane insert does not need any external actuation by the user to function. The sterile barrier is comprised of a frame, in which passages are created, allowing flow through a frame structure. Attached to the top of frame are a plurality of hydrophobic and/or hydrophilic sterilizing grade membranes. A combination of hydrophilic and hydrophobic membranes allows both water and gasses to flow through the assembly while the device is in use. As the devices are stacked, another device may be placed and compressed. The opposing membrane inserts, disposed between the devices, create an isolation region, in which any bioburden, e.g., micro-organisms, bacteria, etc., that contaminated a surface during assembly will be entrapped between opposing membrane inserts. This mechanism of entrapment will prevent the bioburden from reaching other areas of the device during use because the bacteria cannot travel through any of the membranes that comprise this isolation region. Furthermore, the amount of fluid leaking from the device is greatly reduced when the user removes the device from the holder. Turning now to the figures, FIG. 1 depicts a front, perspective view of a connector 100, according to some embodiments of the disclosure. The connector 100 comprises a housing 101 for creating a bi-modal membrane insert, a bi-modal insert being defined as having a hydrophobic and a hydrophilic membrane, which can allow flow of liquids and gases. As shown, the housing 101 is generally circular in shape although it need not be. In fact, the connector 100 merely needs to fit into a complementary shape within a filtration cassette, as discussed more below. In some embodiments, the housing 101 comprises an arcuate top area 115 having an outer rail 105 and an arcuate lower area 111 that comprises an inner rail 106, wherein the arcuate lower area 111 generally has a smaller diameter than the arcuate top area 115. The housing 101 further comprises, optionally, a plurality of legs 109, which extend downward and connect the arcuate top area 115 and the arcuate lower area 111. The housing 101 comprises an upper groove (described below) and a lower groove (described below) within the arcuate top area 115, which locate a first seal 102 and a second seal 102 that is lower than the first seal 102. The first and second seals 102 may be, for example, elastomeric or polymeric o-rings that are compliant.

The arcuate top area 115 houses a plurality of membranes 103, 104. In practice, there can be more than two membranes 103, 104. The two membranes 103, 104, as shown, are concentric and can be the same type of membrane or differing. In at least one embodiment, the membrane 103 may be a hydrophilic, sterilizing-grade membrane, i.e., generally being described as having a pore size rating of approximately 0.22 microns or less, and the membrane 104 may be a hydrophobic, sterilizing-grade membrane and, in some embodiments, both membranes could comprise both hydrophilic and hydrophobic properties. In some embodiments, the membrane(s) 103, 104 is/are capable of passing a suitable bacterial retention test, irrespective of pore size. The connector 100 is capable of being sterilized, wherein the connector 100 is also capable of creating and/or maintaining an aseptic condition.

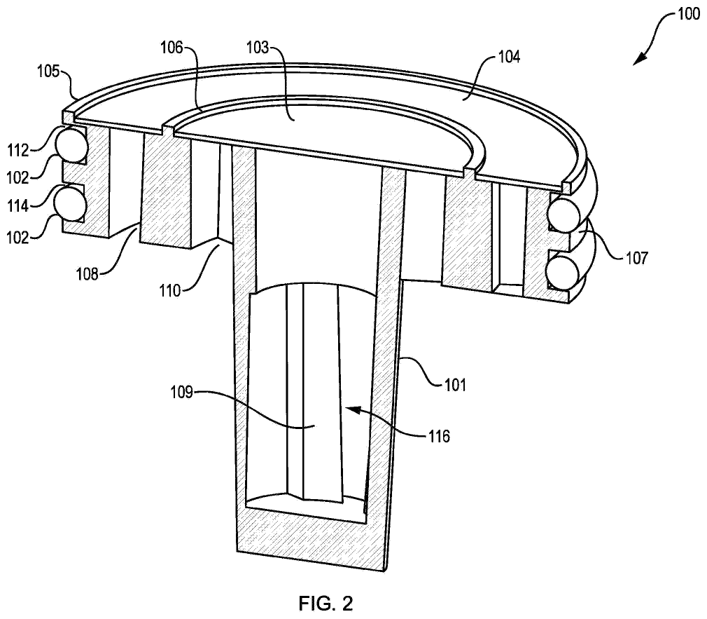

FIG. 2 is a cross-section view of a connector 100 of FIG. 1, according to some embodiments of the disclosure. As shown, the housing 101 further comprises the upper groove 112 and the lower groove 114, which are separated by a groove rail 107. As depicted, the first seal and the second seals 102 are disposed in the upper groove 112 and the lower groove 114. As depicted, the upper groove 112 and the lower groove 114 may be rectangular though optionally are formed as circular. The membranes 103, 104 are separated by the inner rail 106. The membrane 104 is placed between the outer rail 105 and the inner rail 106. An outer channel 108 between the inner rail 106 and the outer rail 105 separates a fluid flow from an inner channel 110. A central channel 116 is an area for fluid flow travelling through a central area in fluid communication with the inner channel 110.

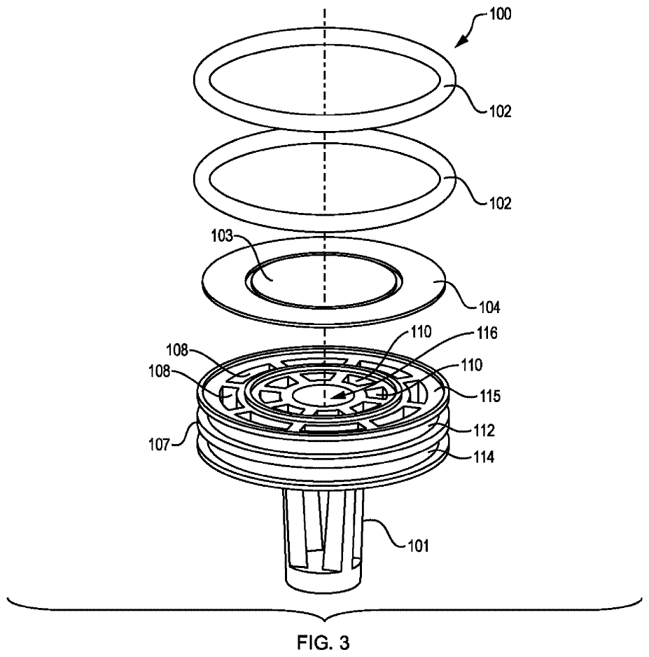

FIG. 3 is an exploded view of a connector 100 of FIGS. 1-2, according to some embodiments of the disclosure. The housing 101 is shown having empty upper groove 114 and upper grove 112 separated by rail 107 and a plurality of outer channels 108 and inner channels 110 and a central channel 116. Also shown are the two seals 102 and the two membranes 103, 104. It is to be understood that a fluid, such as a biological fluid, travels through central channel 116 and/or inner channel 110, to be filtered as it travels membranes 103, 104. In some embodiments, a cassette might have from three to six of connectors 100. For example, a cassette having two inlets, two outlets, and two vents would comprise six connectors 100.

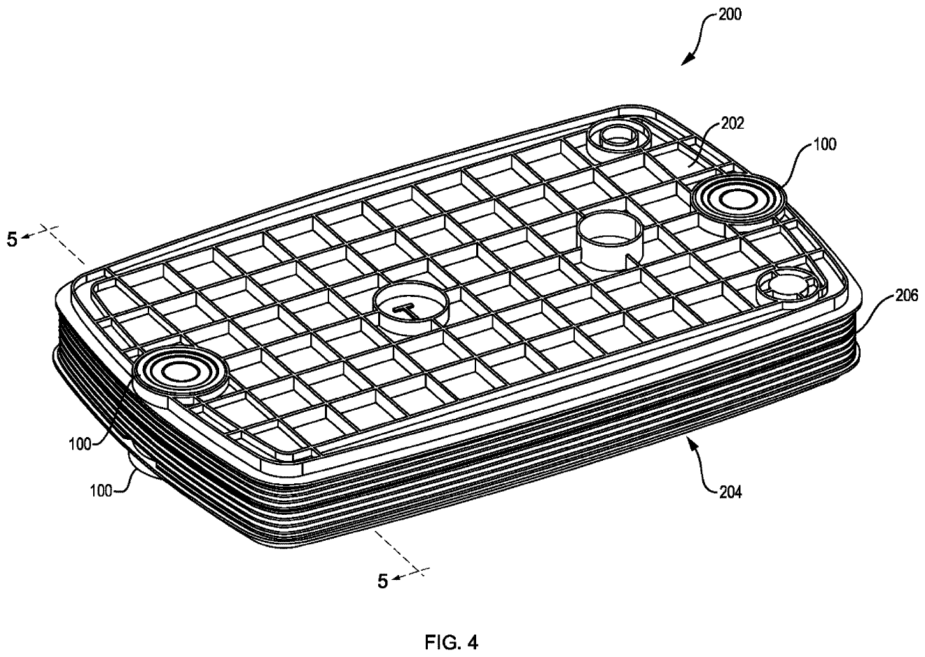

FIG. 4 is a front perspective view of a filtration cassette 200 that can be connected to an adjacent filtration cassette 200 (not shown) via the connector(s) 100 of FIGS. 1-3, according to some embodiments of the disclosure. As shown, there are two connectors 100 on a top side 202 of the filtration cassette 200. Another connector 100 is shown on a bottom side 204 of the cassette. It is to be understood that a fourth connector 100 is also present, although not shown, on the cassette 200. It is to be further understood that two, three, four, five, six, seven, eight . . . and many more cassettes 200 can be joined in any commercially reasonable number together into a single filtration system using, for example, a press. It is to be further understood that any cassette 200 may comprise a plurality of stacked plates 206, each of which can house a filtering membrane(s) and/or a plurality of filtering membranes.

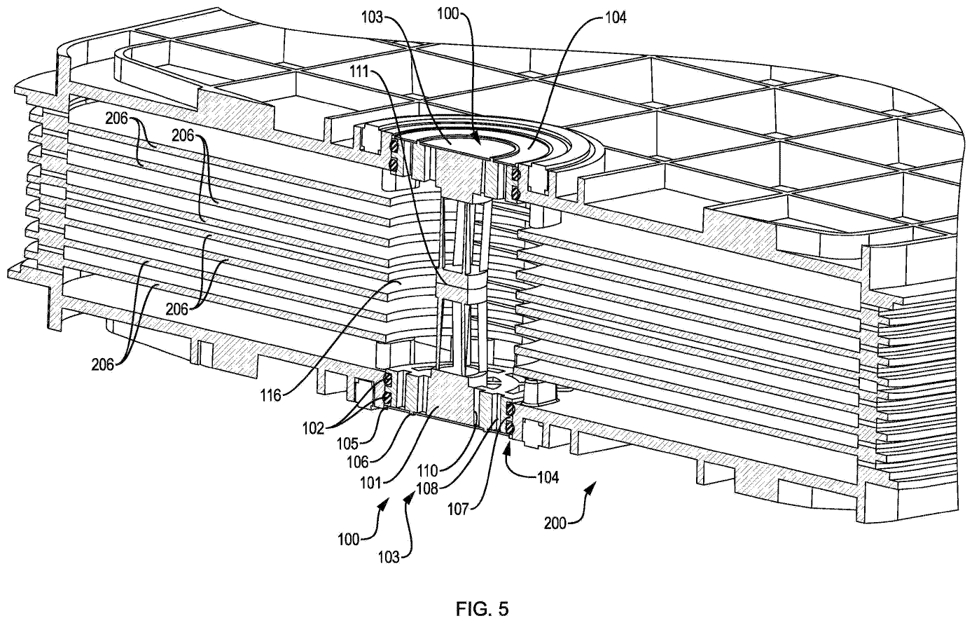

FIG. 5 is a cross-section view taken along line 5-5 of a cassette 200, according to some embodiments of the disclosure. As depicted, the cassette 200 has two connectors 100 in fluid communication with each other. As can be seen, the lower arcuate area 111 of the connectors 100, placed in each of the cassettes 200, butt up against each other. Also shown are a plurality of filtering membranes 206, surrounding the central channel 116. The other features are also shown for context, including the housing 101, the rail 107 and a plurality of outer channels 108 and inner channels 110 and a central channel 116. Also shown are the two seals 102 and the two membranes 103, 104, and the outer rail 105, and the inner rail 106.

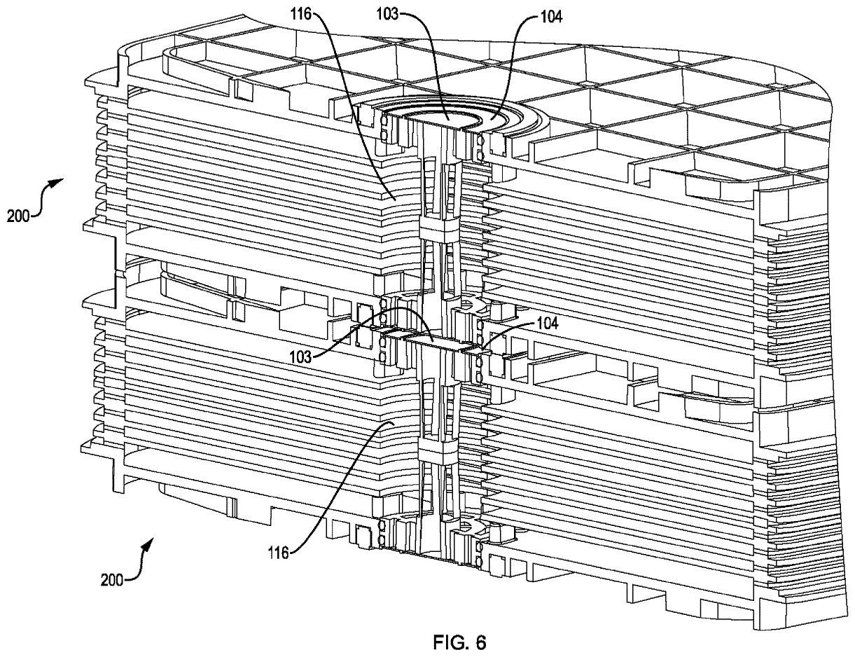

FIG. 6 depicts cross-section views of two cassettes 200, wherein the two cassettes 200 are stacked via connectors 100, according to some embodiments of the disclosure. It is to be understood that when two or more cassettes 200 are stacked, they are in fluid communication via the connectors 100. The cassettes need not be joined except by application of pressure to be in fluid communication. It is to be further understood that the connectors 100 can be placed within any inlet, outlet, or vent of any of the cassettes 200. Alternatively, a plug (not shown) can be placed in one or more of a plurality of filtration cassettes 200. The cassettes 200 can be stackable devices having filtering membranes. For example, the cassettes 200 can include, e.g., Pellicon®, Pellicon®2, Pellicon®3, Natrix® devices, depth filtration cassettes, such as Millistak+® devices, tangential flow filtration cassettes, such as Prostak™ devices, Viresolve® Pro Magnus Device, marketed by the EMD Millipore Corporation, Burlington, MA, USA, which embody a cassette-style format. Natrix® devices that are in cassette-style formats, such as cassette 200, may comprise a macroporous cross-linked gel, wherein the macroporous cross-linked gel comprises a polymer formed from a reaction of one or more polymerizable monomers with one or more cross-linkers; the macroporous cross-linked gel is located within the pores of a support member; and macropores of the macroporous cross-linked gel are smaller than the pores of the support member. In some embodiments, the composite material is for bio-affinity chromatography. In some embodiments, the composite material is a porous membrane. In some embodiments, the composite material may be a porous polymeric gel membrane. In some embodiments, a manifold-free filtration system, comprising two or more adjacent cassettes in fluid communication with each other via a plurality of connectors, is disclosed.

Embodiment Two: Linear Single Use Elastomeric Aseptic Valve. The linear single use elastomeric aseptic valve embodiment describes a low-profile, genderless valve that can be integrated into a stackable cassette style device. The linear single use elastomeric aseptic valve embodiment enables a flow path of a plurality of stacked devices, such as those discussed above, to be opened in an aseptic manner, without the need for external manifolds. In addition, the linear single use elastomeric aseptic valve allows the flow path of each of the plurality of stackable devices/cassettes to be isolated upon disassembly. For example, some of the described connections can be reversed to close the device. Each cassette can be removed from a holder in a substantially dripless manner. The holder comprises a top plate and a bottom plate, connected by rods. A hydraulic piston or other hydraulic device compresses the holder and therefore the cassettes between the top plate and bottom plate, creating a seal between the devices. For example, the Viresolve® Pro Magnus Holder, as marketed by the EMD Millipore Corporation, Burlington, MA, USA. Other holders are described in WO2022108902, which is incorporated in its entirety. The linear single use elastomeric aseptic valve comprises a body, and two complementary valve halves, which houses an elastomeric (or otherwise suitably compressible or compliant material(s)) slide having a sterile orifice, along with a solid section. Therefore, the flow path can be aseptically isolated in a closed position. The elastomeric slide can be constructed by, for e.g., disposing or otherwise overmolding an elastomeric material, for e.g., silicone, a thermoplastic elastomer, a thermoplastic polyolefin, and the like, onto a frame, which is a thermoplastic or thermoset material in some embodiments, that gives the elastomer support and/or rigidity. A film or peel strip may be placed over the elastomeric slide, adjacent to the flow path, which can be removed prior to use. The linear genderless valve is capable of being sterilized and preventing the proliferation of pathogens, i.e., wherein the genderless valve is capable of creating and/or maintaining an aseptic condition. The film may be made of a polymeric material, a plastic film, a plastic sheet, etc. The polymeric material may be, e.g., a nylon, a polyester, polyolefins, etc. The film may also comprise a metalized polymeric film, for e.g., a plastic film coated with aluminum. In some embodiments, the film may be a metal film coated with a polymeric material. In some embodiments, the film may be a metal film. The film creates a sterile barrier for the sterile orifice before use. The sterile orifice may be sterilized by steam treatment, gamma irradiation, x-ray, ethylene oxide, or other sterilizing treatments as are known to those in the art. A solid end of the elastomeric slide at least partially covers the flow path, preventing contamination. As the cassettes/devices are stacked and joined or otherwise placed in fluid communication, the two valve halves come together and are compressed in place. The elastomeric slide comprises handles. Both handles are pulled to open the flow path between two adjacent devices/cassettes, which can be done simultaneously or separately. During this pulling motion, the sterile orifices on each side of the elastomeric slide pull out from the films and align with the flow path in the housing of the elastomeric slide. In other words, a genderless valve may comprise a body, a handle, wherein the handle comprises a closed side and an open side, a port attached to the body, an elastomeric slide attached to the handle, wherein the elastomeric slide comprises an orifice, and a film sealed or releasably attached to the elastomeric slide. Optionally, a second genderless valve having a second orifice, wherein the orifice and the second orifice may be in fluid communication with one another genderless valve. As the orifices of two genderless valves align, optionally, a ribbed feature, e.g., molded into the housing compresses the orifices against each other, forming a hermetic seal, i.e., a hermetic seal between the housings and sterile orifices. A second compression of the stacked devices/cassettes may optionally be applied by a holder to further compress the hermetic seals, creating an even greater robust seal. The device's/cassette's flow paths are now open, and the user can use the devices/cassettes to process a fluid. Once processing is complete, the user may then remove a portion of the compression from the devices. The valves may then be pushed by the handles into a closed position. This motion covers the flow path once more using the elastomeric valve. This action, i.e., manipulating the valve from open to closed, isolates the flow path, substantially preventing residual fluid from dripping out of the device. The user can then finish decompressing fluid pressure and decompressing the holder before removing and discarding the used devices/cassettes.

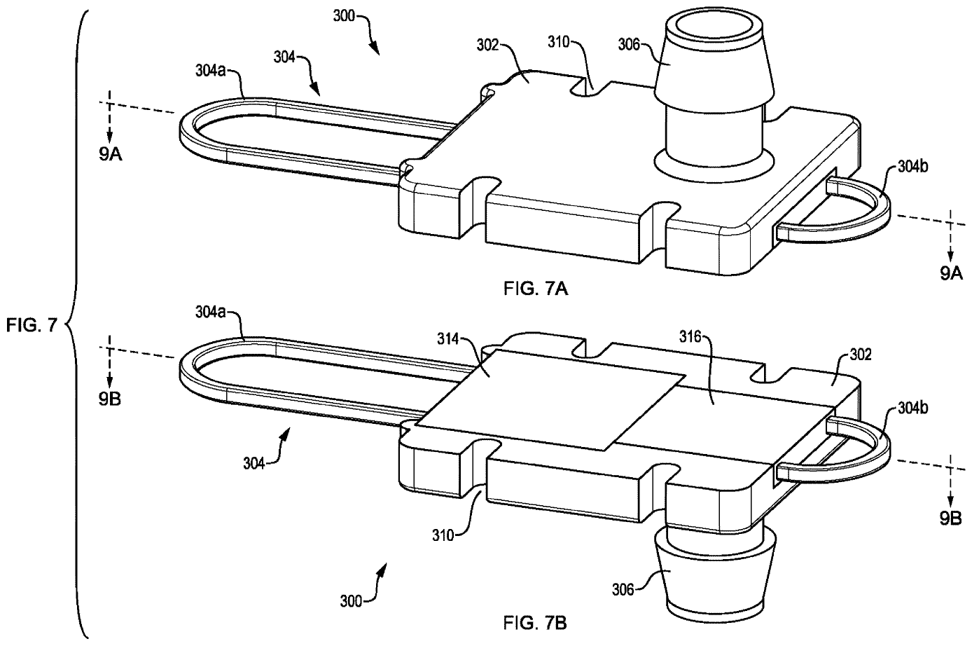

FIG. 7 is a front perspective view of a valve 300, according to some embodiments of the disclosure. FIG. 7 comprises FIG. 7A of the valve 300 and FIG. 7B, which is a complementary valve 300, which is oriented upside down with respect to the valve 300 of FIG. 7A, i.e., a mirror image. The valve 300 is a genderless valve and comprises a body 302 and a handle 304 having a closed side 304a and an open side 304b. The valve 300 is noted as a genderless valve because neither valve 300 nor a complementary valve 300 has any portion that protrudes into the other in forming a fluid connection. As a fluid travels through a port 306 of one valve 300 and out the port 306 of a complementary valve 300, the two valves 300 are held together by pressure from, for e.g., a hydraulic press. As shown, the valve 300 in FIGS. 7A-7B are in the closed position. The valve 300 further comprises a port 306 and, optionally, a plurality of body notches 310. As shown in FIG. 7B, the valve 300 further comprises an elastomeric slide 316 and a film 314. The film 314 is attached to the elastomeric slide 316. The elastomeric slide 316, which is attached to the handle 304, is made of, for example, a silicone material or a thermoplastic elastomer. As shown below, the elastomeric slide 317 comprises a valve orifice (shown more clearly below).

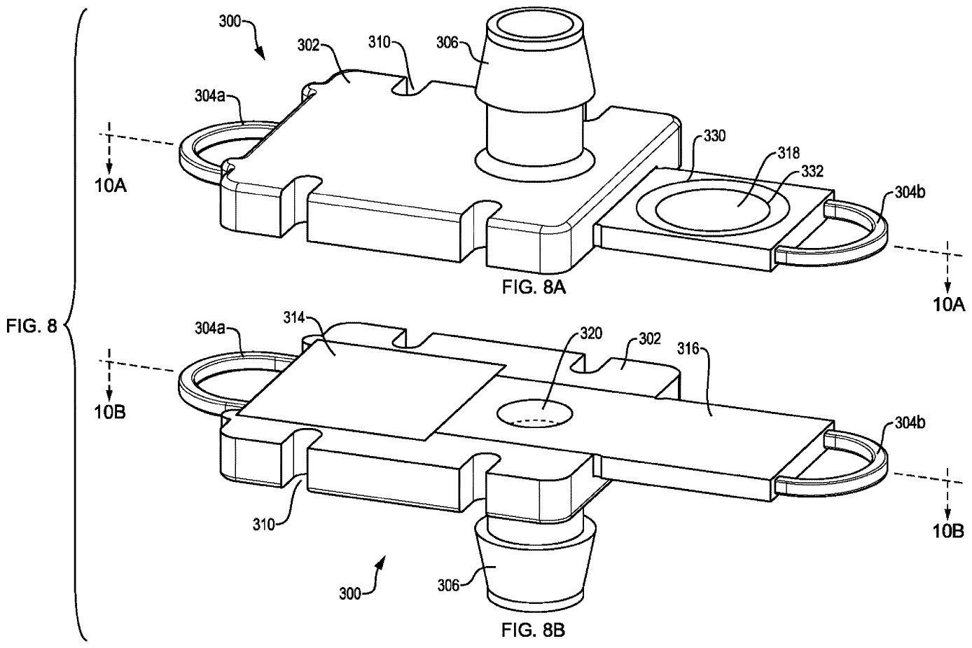

FIG. 8 depicts the valve 300 having a complementary valve 300 associated therewith, as in FIGS. 7A-7B, but here in an open position as depicted in FIGS. 7C-7D. The elastomeric slide moves linearly when a force is applied to the handle, i.e., pulling or pushing the open side handle 304b biases the elastomeric slide 316 away from the film 314 in a linear fashion, exposing a valve orifice 320. Delivery of a fluid, such as a biological fluid, to the port 306 of the valve 300 of FIG. 7C, allows the fluid to flow therethrough, e.g., through the valve orifice 320 in both the valves in 300, and out the port 306 of valve 300 in FIG. 7D, if both are in the open position, forming a flow path. The film 314 is a sterile barrier, protecting the sterility of the valve orifice 320, which might be sterilized via steam, x-ray, gamma-irradiation, ethylene oxide treatment, ozone-treatment and/or other sterilizing treatments as are known to those in the art. A port side of the slide 318 may also comprise one of more chamfered edges. For example, a first chamfered edge 330 and/or a second chamfered edge 332.

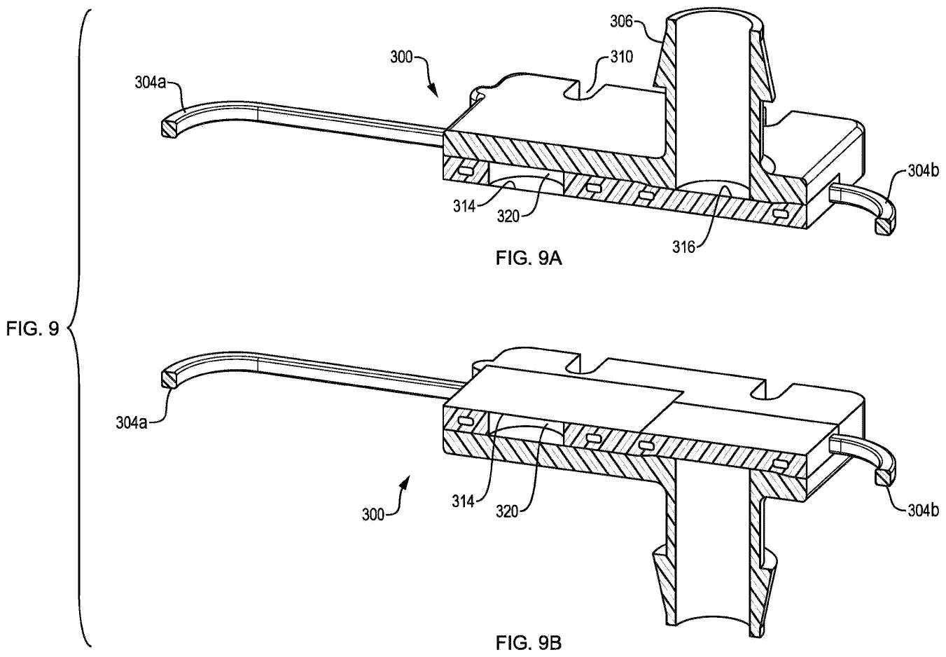

FIG. 9 is a cross-section view taken along line 9-9 of the valve 300 of FIG. 7A and further including a complementary valve 300 in closed positions, according to some embodiments of the disclosure. As depicted in FIG. 9, each of the valves 300 comprise a sterile orifice 320 in the closed position, and each having the film 314 disposed thereon.

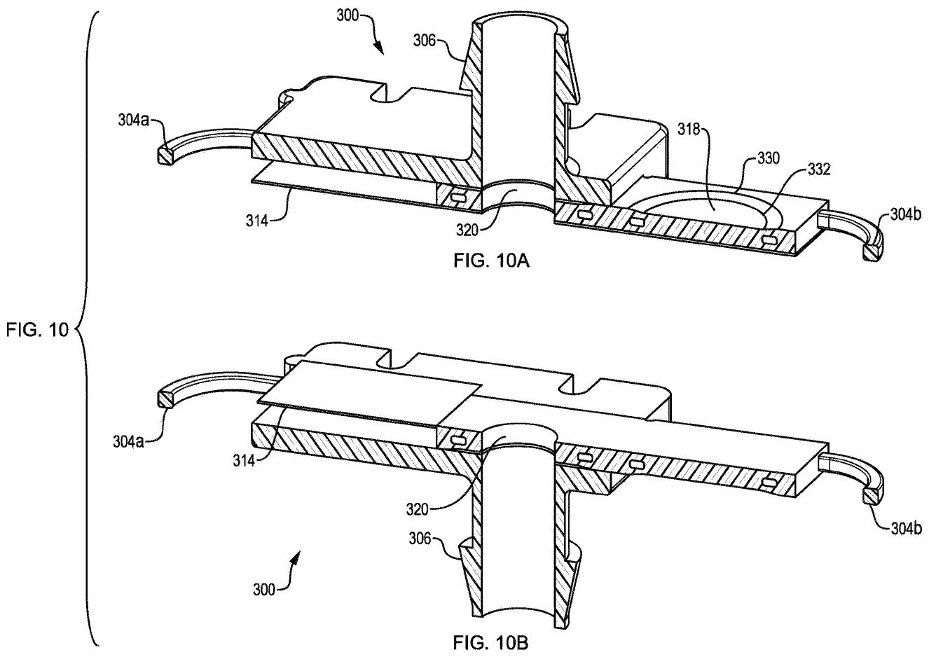

FIG. 10 is a cross-section view taken along line 10-10 of a valve 300 of FIG. 7D and further including a complementary valve 300 in open positions, according to some embodiments of the disclosure. As depicted in FIG. 10, each of the valves 300 comprise a sterile orifice 320 in the open position, and each having the film 314 removed therefrom, creating an open valve condition, wherein a liquid supplied to either port 306 could flow therethrough.

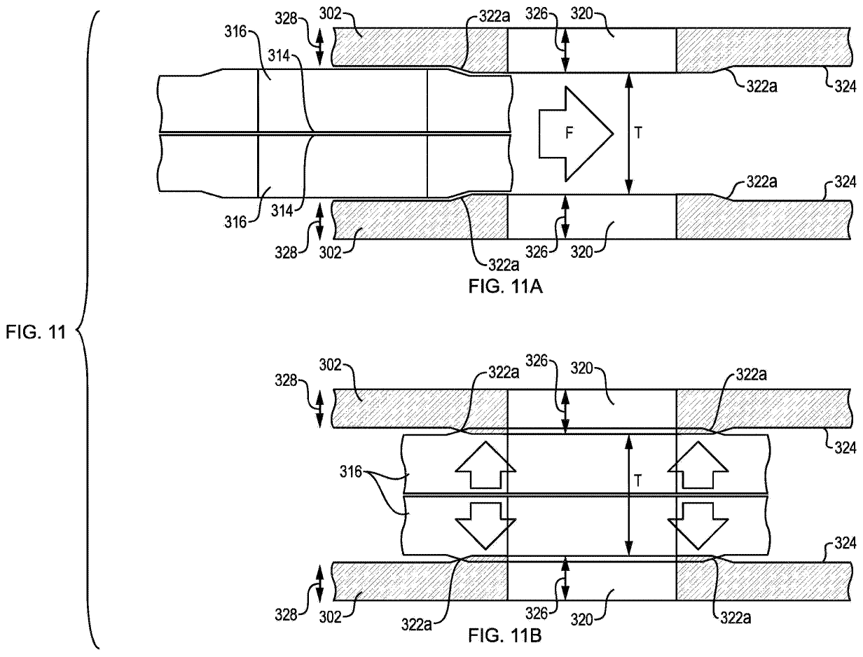

FIG. 11 depicts a close view of the cross-sections of FIGS. 9-10, according to embodiments of the disclosure. FIG. 11A depicts a close up of two valves 300 in closed positions. FIG. 11B depicts a close up of two valves in open positions. The body 302 of the valves 300 are shown. Each of the body 302 of each valve 300 has internal surfaces 324. The internal surfaces 324 each have two chamfers 322a, which neck down to a larger body thickness 326 near the valve orifice 320 as compared with a smaller body thickness 328 on a perimeter of the body 302. Because a thickness T adjacent the valve orifices 320 is smaller, an interference fit is created when the handles (not shown) are pulled in a direction F. In other words, the two elastomeric slides 316 fit into a smaller space, providing a tighter seal and promoting a leak-free condition.

Embodiment Three: Rotary Elastomeric Aseptic Valve. The rotary elastomeric aseptic valve embodiment is similar to the linear single use elastomeric aseptic valve embodiment. The rotary elastomeric aseptic valve is actuated via rotary manipulation of a handle, as opposed to a linear fashion. The rotary actuation reduces the force needed to actuate the valve due to the inherent mechanical advantage of a rotary design. In addition, the rotary elastomeric aseptic valve requires much less space on an end cap when integrated within a device/cassette, at least because linear motion is eliminated. The mechanisms of sealing and maintaining sterility of the flow paths and orifices are substantially similar. The rotary elastomeric aseptic valve, which is also genderless, is capable of being sterilized, wherein the genderless valve is capable of maintaining an aseptic condition.

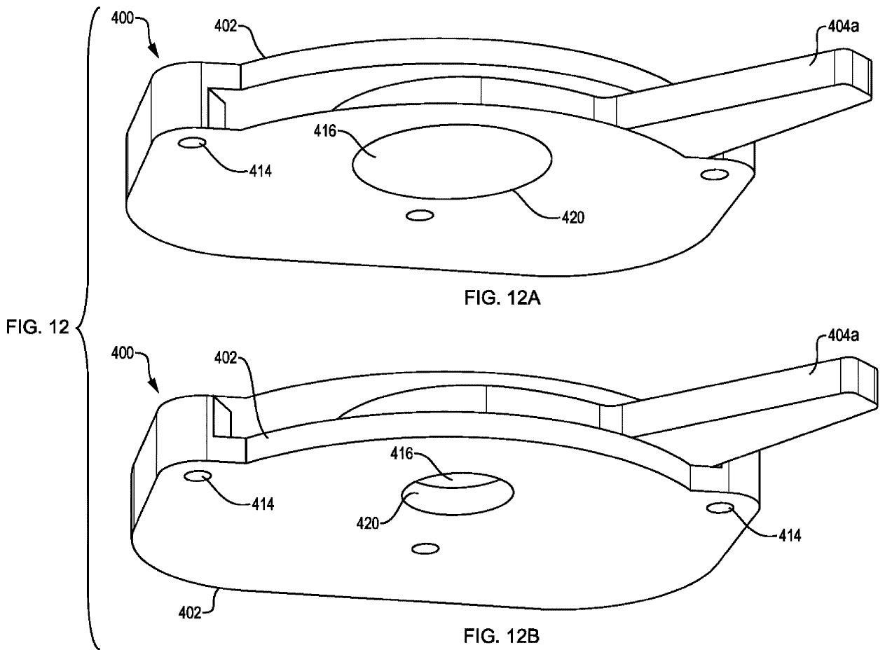

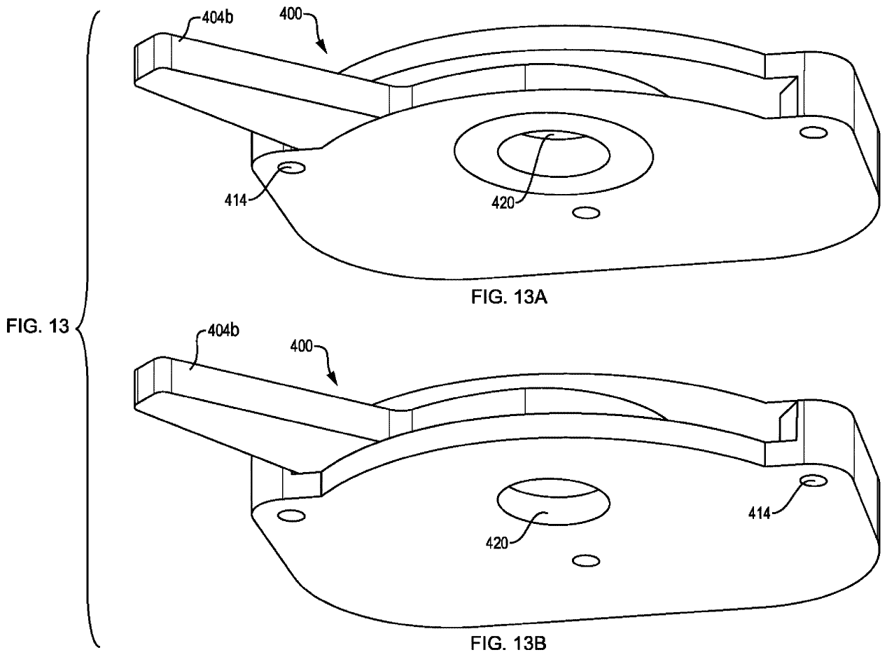

FIG. 12 depicts FIGS. 12A-12B showing two complementary rotary elastomeric aseptic valves 400 in closed positions, according to some embodiments of the disclosure (also referred to as rotary valves 400). FIG. 12A depicts a bottom perspective view of the rotary elastomeric aseptic valve 400. FIG. 12A depicts a top perspective view of the rotary elastomeric aseptic valve 400. The two rotary elastomeric aseptic valves 400 are complementary because they work together to form a channel or circuit for a fluid to flow therethrough, similar to the linear, genderless, complementary valves discussed above. For example, through a channel formed by the rotary valve orifice 420, as discussed more fully below. The rotary valve 400 is a genderless valve and comprises a body 402 and a handle 404 having a closed side and an open side. As shown, the valves 400 in FIGS. 12A-12B are in the closed position, wherein the handle 404 is shown as 404a. In FIG. 12B, a lower surface of the rotary slide 416 can be seen. Alignment holes 414 allow the attachment of valves 400 and are optionally provided within the body 402 and a body cover 420. Other attachment means are possible, such as gluing, ultrasonic welding, overmolding, RF welding, and other attachment means as known to those of skill in the art. Also, the holes 414 aid the alignment of the body 402 with a body cover 420. FIG. 13 depicts two rotary valves 400 in open positions, according to some embodiments of the disclosure. As shown, the handle 404 is in position 404b, which is opposite that of 404a, shown above. The open position is easily seen as a through hole can be witnessed by rotary orifice 420 in FIG. 12C.

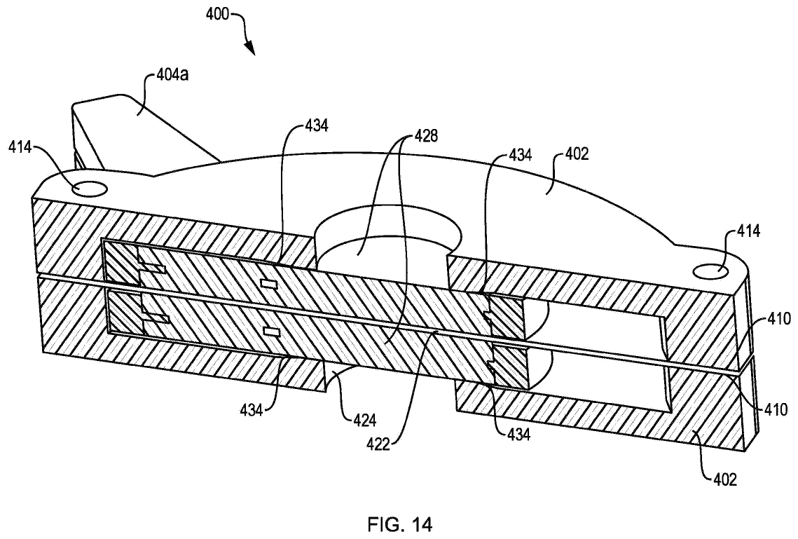

FIG. 14 depicts FIGS. 12A-12B showing a cross-section of two complementary rotary elastomeric aseptic valves 400 in closed positions in an assembled view, according to some embodiments of the disclosure. As can be seen, the handle 404 is in the closed position 404a. The rotary slide 428 therefore blocks any fluid that might otherwise travel through to the other rotary aseptic valve 400. Also, each of the body 402 further comprises at least one internal chamfer or other transitional edge 434 near the body hole 424 so that the rotary slide 428 is allowed to slide but remains tight to the body 402.

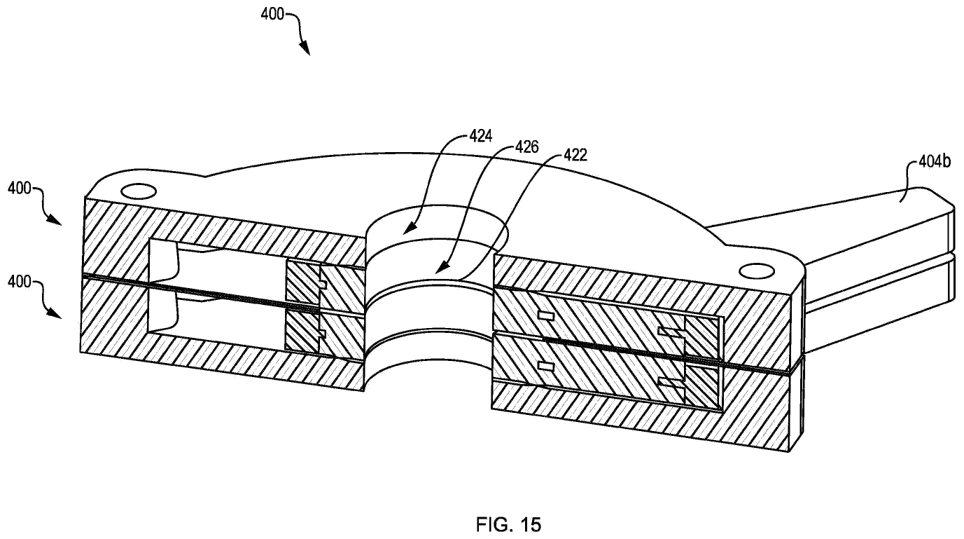

FIG. 15 depicts FIGS. 12C-12D showing a cross-section of two complementary rotary elastomeric aseptic valves 400 in open positions in an assembled view, according to some embodiments of the disclosure. As can be seen, the handle 404 is in the open position 404b. The rotary slide 428 is therefore in a position allowing any fluid that might travel through to the other rotary aseptic valve 400. Specifically, the body hole 424, a rotary slide hole 426 and the cover hole 422 are aligned in both the two rotary valves 400. It is to be understood that many rotary valves 400 may be pressed, assembled, or joined together with a filtration system, as with the linear valve 300 discussed above.

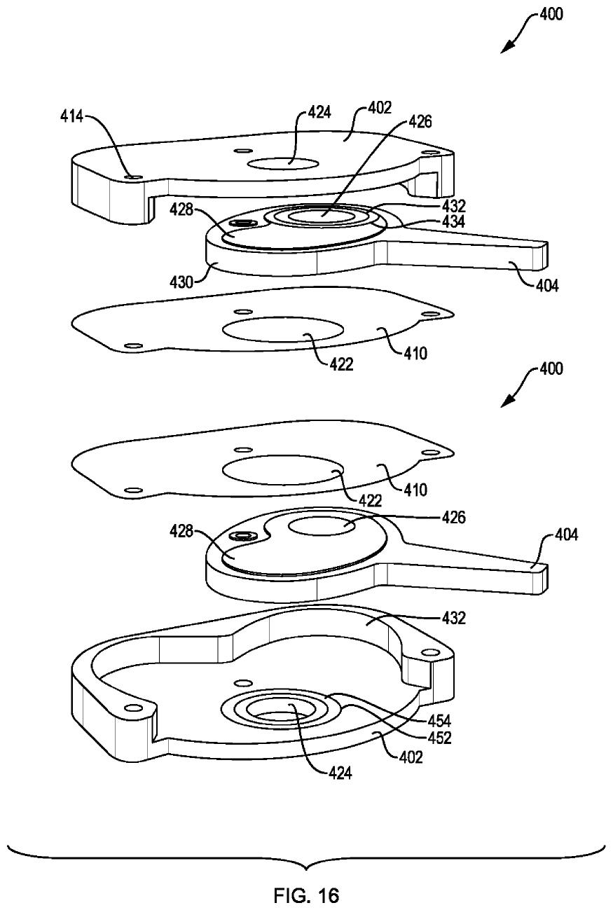

FIG. 16 depicts an exploded view of the two rotary valves 400 of FIGS. 12-15, according to some embodiments of the disclosure. It is to be understood that the two rotary valves 400, as depicted, are mirror images of each other. As shown, the exploded view shows each of the rotary valves 400 in closed positions. Each of the rotary valves 400 comprises the body 402, having a body hole 424; wherein the handle 404 comprises the rotary slide 428 or is otherwise attached thereto, and the rotary slide hole 426; and a body cover 410, which comprises a body cover hole 422. The body cover hole 422 and the body hole 424 and substantially concentric when the rotary valve 400 is assembled. The handle 404, having the rotary slide 428 and slide hole 426 is arranged such that when the rotary valve 400 is assembled, the slide hole 426 is not concentric with at least one of the body hole 414 or the body cover hole 422, wherein the handle 404 is in a closed position. When the handle 404 is in an open condition, the slide hole 426, the body hole 414 and the body cover hole 422 are substantially concentric, allowing a channel or circuit therethrough for a fluid. The handle 404 can be made of any suitable material, for example, a thermoplastic. As shown, the rotary slide 428 comprises an arcuate edge 430 so that it may rotate, at least partially, freely with an edge 432 of the body 402 when switching from a closed position to an open position or vice versa. The rotary slide 428 may also comprise a raised area having chamfered edges 432, 434 adjacent the rotary slide hole 426. Similarly, the rotary body 402 may comprise internal body chamfers 452, 454 adjacent the body hole 424. The rotary slide 428 can be made of any suitable compliant material, such as a thermoplastic elastomer or silicone. In some embodiments, the rotary slide 428 is thin such that an air gap (as is described more fully below) is created between the rotary slide 428 and the body cover 410. For example, in some embodiments, the air gap is approximately 0.125 centimeters (cm) or, for e.g., 0.020-0.180 cm in some embodiments, or 0.010-0.100 cm in some embodiments; or 0.030-0.090 cm, in some embodiments. It is believed that any contaminants on the surface of the rotary slide 428 will not be transferred to another area of the rotary valve 400, owing to the air gap, during actuation of the rotary valve(s) 400. Typically, it is the rotary slide 428 that is thinner to create the air gap.

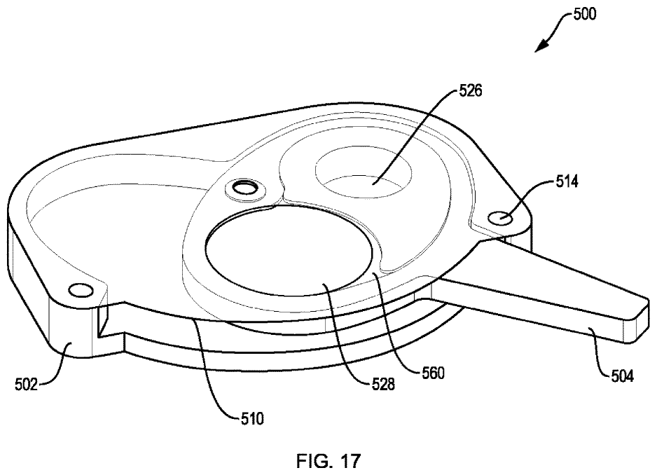

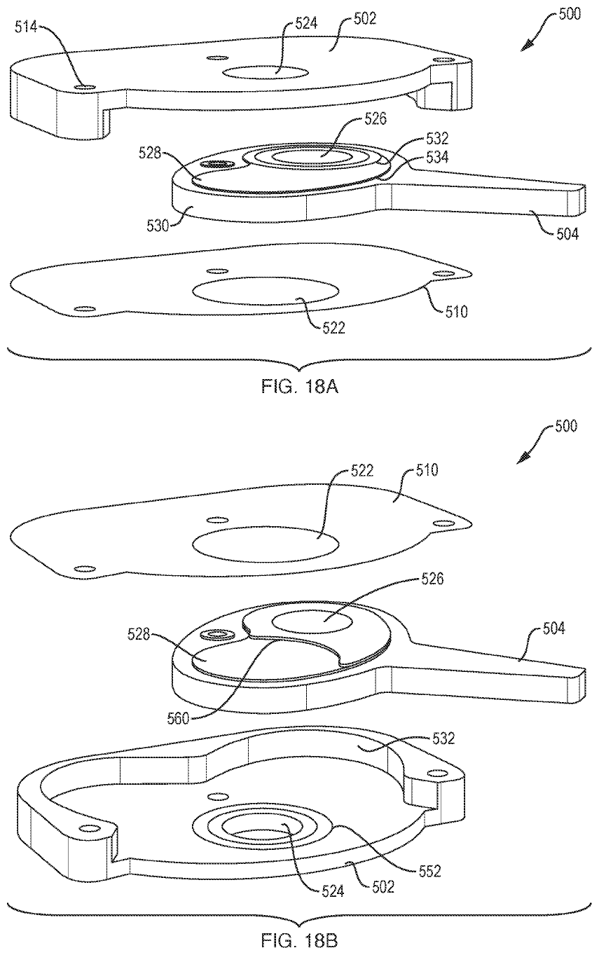

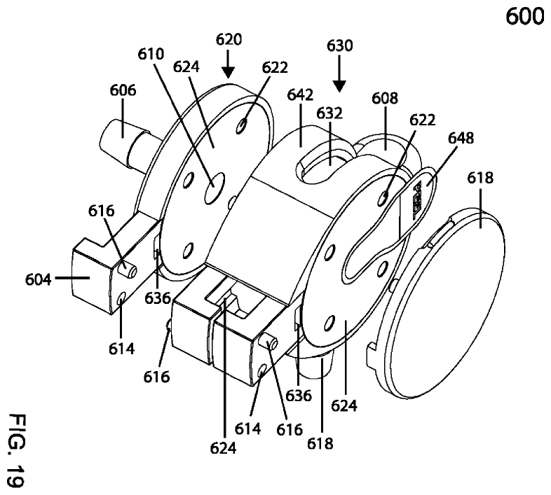

FIG. 17 depicts a rotary valve 500 having an air gap 560, according to some embodiments of the disclosure. The rotary valve 500 is similar to the rotary valve 400. The rotary valve 500 is genderless. In other words, genderless indicates that no part of one rotary valve 500 receive any part of another rotary valve 500 to which it is in fluid communication. In FIG. 17, the body cover 510 is shown as transparent although this need not be the case. The body cover 510 may be made of a metal, such as stainless steel, or any suitable polymeric material, thermoplastic elastomer, or thermosetting material, whether transparent, translucent, or opaque. In this context, suitable indicates that the polymeric material is capable of being sterilized while remaining robust for all bioprocessing applications. For example, parts of the rotary valve 500 could be sterilized via at least one of the following: ethylene-oxide treatments, corona treatments, steaming, gamma radiation, ethyl alcohol, hydrogen peroxide gas, x-ray, or other sterilizing treatments known to those in the art. The air gap 560 is a vertical distance created by the differing heights of the rotary slide 528 and the chamfered edges 532, 534 (shown below in FIG. 18) and/or the body cover 510. In some embodiments, the rotary slide 528 is thin such that an air gap 560 is created between the rotary slide 528 and the body cover 510. For example, in some embodiments, the air gap 560 is approximately 0.125 centimeters (cm) or, for e.g., 0.020-0.180 cm in some embodiments, or 0.010-0.100 cm in some embodiments; or 0.030-0.090 cm, in some embodiments. It is believed that any contaminants on the surface of the rotary slide 528 will not be transferred to another area of the rotary valve 500, owing to the air gap, during actuation of the rotary valve(s) 500. Typically, it is the rotary slide 528 that is thinner to create the air gap 560.

FIG. 18 depicts an exploded view of two rotary valves, the rotary valve 500 as shown in FIG. 17, according to some embodiments of the disclosure. It is to be understood that the two rotary valves 500, as depicted, are mirror images of each other. As shown, the exploded view shows each of the rotary valves 500 in closed positions. Each of the rotary valves 500 comprises the body 502, having a body hole 524; wherein the handle 504 comprises the rotary slide 528 or is otherwise attached thereto, and the rotary slide hole 526; and a body cover 510, which comprises a body cover hole 522. A port (not shown) would typically be attached, integrally formed and otherwise in fluid communication with the body hole 524. The body cover hole 522 and the body hole 524 and substantially concentric when the rotary valve 500 is assembled. The handle 504, having the rotary slide 528 and the slide hole 526 is arranged such that when the rotary valve 500 is assembled, the slide hole 526 is not concentric with at least one of the body hole 514 or the body cover hole 522, wherein the handle 504 is in a closed position. When the handle 504 is in an open condition, the slide hole 526, the body hole 514 and the body cover hole 522 are substantially concentric, allowing a channel or circuit therethrough for a fluid. The handle 504 can be made of any suitable material, for example, a thermoplastic. As shown, the rotary slide 528 comprises an arcuate edge 530 so that it may rotate, at least partially, freely with an edge 532 of the body 502 when switching from a closed position to an open position or vice versa. The rotary slide 528 may also comprise a raised area having chamfered edges 532, 534 adjacent the rotary slide hole 526. Similarly, the rotary body 502 may comprise internal body chamfers 552, 554 adjacent the body hole 524. The rotary slide 528 can be made of any suitable compliant material, such as a thermoplastic elastomer or silicone.

FIG. 19 depicts a partially exploded view of a genderless expandable valve 600, according to some embodiments of the disclosure. The genderless expandable valve 600 comprises an inline valve 620 and a center valve 630. The center valve 630 is capable of receiving a cap 618 at a first end 654 that is opposite a second end 656. The inline valve 620 comprises an inlet port 606, such as a barb port. The inlet port 606 may be integrally formed, i.e., a single piece made with one operation, such as an injection molding or 3D-printing process. Alternatively, the port 606 may comprise threads (not shown) for screwing into a corresponding tapped hole (not shown) within the inline valve 620. The inline valve 620 further comprises a slider 604, having a slot 636, as discussed more fully below. A post 616 projects from the slider 604 and has a locating hole 614 to receive a post from, for example, a post 616 on the center valve 630. The center valve 620 further comprises two arcuate members 626, a first arcuate member and a second arcuate member opposite the first arcuate member, as discussed more fully below. In some embodiments, a second or third inline valve 620 and/or center valve 630 can be added to the genderless, expandable valve 600. The first arcuate member and the second arcuate member receive the slider 604 and a valve disk 624 having a disk hole 610, through which liquid flows when the genderless expandable valve 600 is in an open configuration. The valve disk 624 also comprises one or more holes 622 through which a corresponding number of posts from the center valve 630 can penetrate so that the center valve 630 can be assembled. The valve disk 624 is suitably made of any elastic, compliant material, such as a thermoplastic, a thermoplastic olefin, or a thermoplastic elastomer. The disk hole 610 may be covered by a sterile peel strip, as discussed below.

The center valve 630 comprises housing 642. The housing 642 comprises housing slot 632 for allowing a rotary handle 608 to partially rotate therewithin. The rotary handle 608 is joined with or is an integral part of a hub, wherein the hub is housed within the housing 642. For example, the rotary handle 608 may be a separate piece that has an interference fit with a hole in the hub or be screwed into the hub. The hub also accommodates a first hub slider 634 on a second end 656 and a second hub slider 638, opposite the first slider 634 located on the second end 654 of the housing 642 of the center valve 630. As with the inline valve 620, the first slider 634 and the second slider 638 are each held by two arcuate members 626, a first arcuate member and a second arcuate member opposite the first arcuate member (not shown). A disk 624 is also disposed over the first slider 638. The disk 624 may also have a peel strip 648 disposed thereon to keep the surface of the disk sterile until the genderless expandable valve 600 is ready for use. Projecting from the center valve 630 is an outlet port 628 that is generally perpendicular to the inlet port 606.

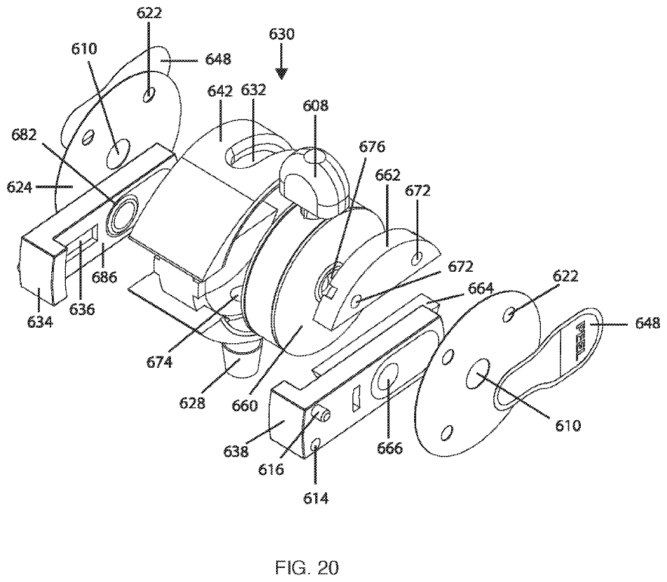

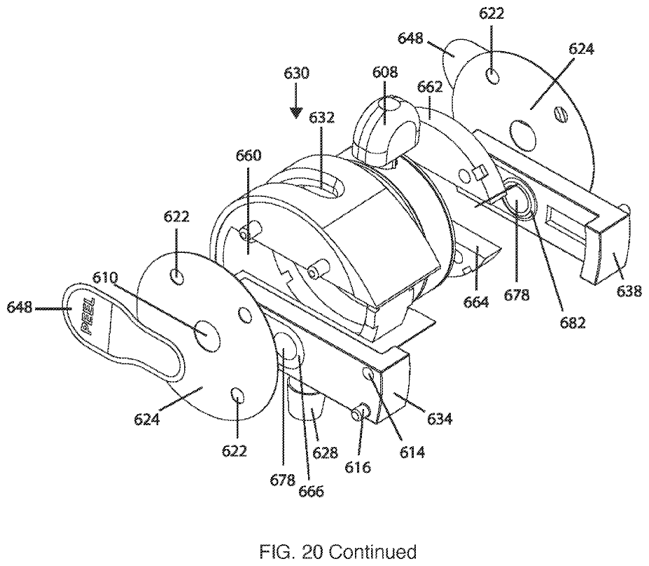

FIG. 20 depicts alternate perspective exploded views of the genderless expandable valve 600 of FIG. 19, absent the inline valve 620, according to some embodiments of the disclosure. In both views, the disk 624 is shown outside the exploded view for ease of understanding. The views show the components of the hub assembly. A first view shows an upper left perspective view of the center valve 630. The slider 634 has slot 636 and a valve hole 678. The valve hole 678 is surrounded by a thermoplastic olefin or thermoplastic elastomer. For example, the valve hole 678 may be surrounded by a silicone rubber. Optionally, the valve hole 678 may transition in height from a higher thickness to a transition area 682 to a lower area 686. The higher thickness and transition area 682 allow the slider 634 to slide more easily from a closed position to an open position while maintaining a leak-free condition while in the open condition. A hole 674 in the center valve 630 leads to the outlet port 628. A hub hole 676 is also shown in a center of the hub 660. The hub 660 having the rotary handle 608 fits inside of the housing 642. The second slider 638 having the hole 678 from the opposite side, showing a flat hole 666 surrounded by an elastomer 684, such as silicone rubber, which may be, for example, over molded into the slider 638. A first arcuate member 662 is shown above the slider 638 and a second arcuate member 664 is shown below the slider 638. Assembly holes 672 are shown to join the sliders 634, 638 with the cap (not shown) and the hub 660. A second view shows an upper right perspective view depicts the same components although showing the opposite surfaces of the components shown in the first perspective view. For example, a backside of the slider 634 depicts the post 616 and hole 614

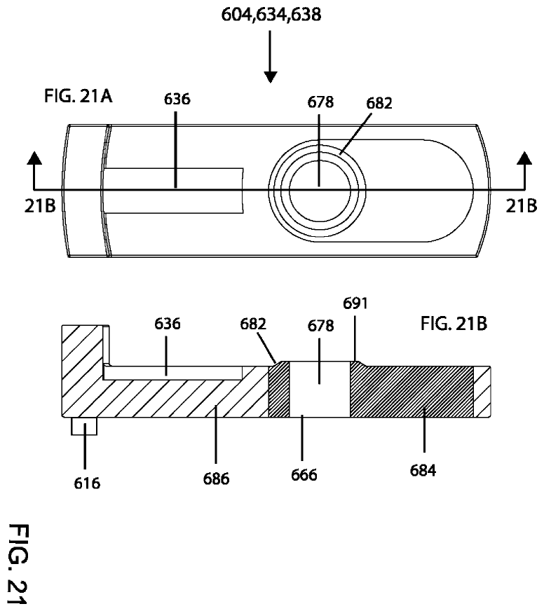

FIG. 21 depicts sliders 604, 634, 638 for use within a genderless expandable valve, according to some embodiments of the disclosure. FIG. 21A shows an upper view of the slider(s) 604, 634, 638 and shows the slot 636, which locates a pin or a post (not shown) and allows the pin or post to slide within the valve 600 and prevents the sliders 604, 634, 638 from being removed from the valve 600 during operation. The hole 678, surrounded by the transition area 682, is also shown. The transition area 682 is a stepped area wherein the surface of the sliders 604, 634, 638 projects upward to make a seal with a similar area of a corresponding slider 604, 634, 638. The silicone may be insert-molded, overmolded, two-shot molded, transfer-molded, liquid injection-molded, or cast, or via other plastics operations as is known to those in the art. For example, the area shown in cross-section may be a polymeric material and formed via injection-molding. Thereafter, a silicone material may be injected into the area shown to produce the slider(s) 604, 634, 638. In FIG. 21B, a cross-section shows the transition area 682, which steps up from a lower surface 686 of the slider(s) 604, 634, 638 to a higher surface 691. The flat side 666 of the hole 678 is opposite the higher surface 691. Also shown is the post 616.

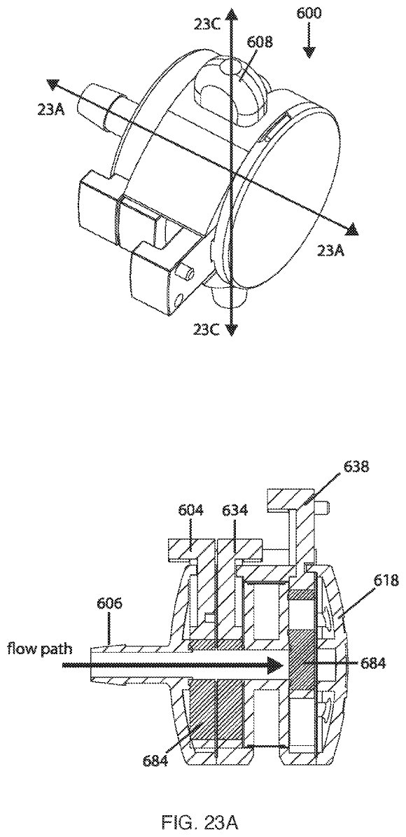

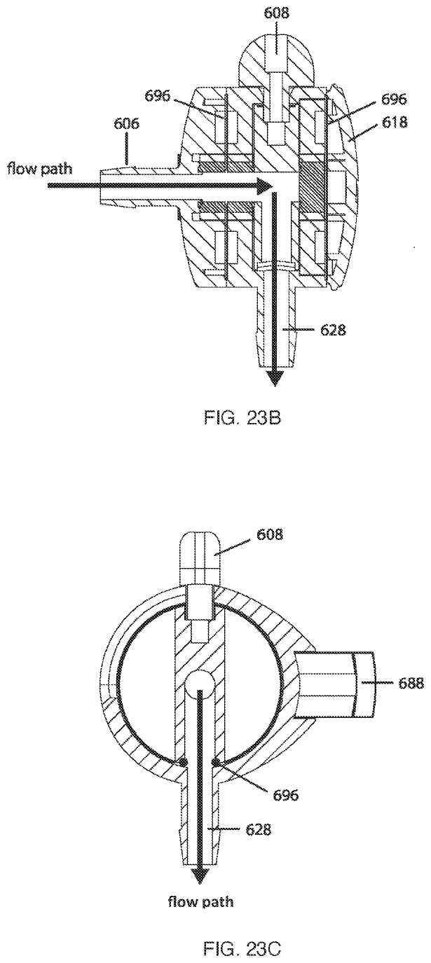

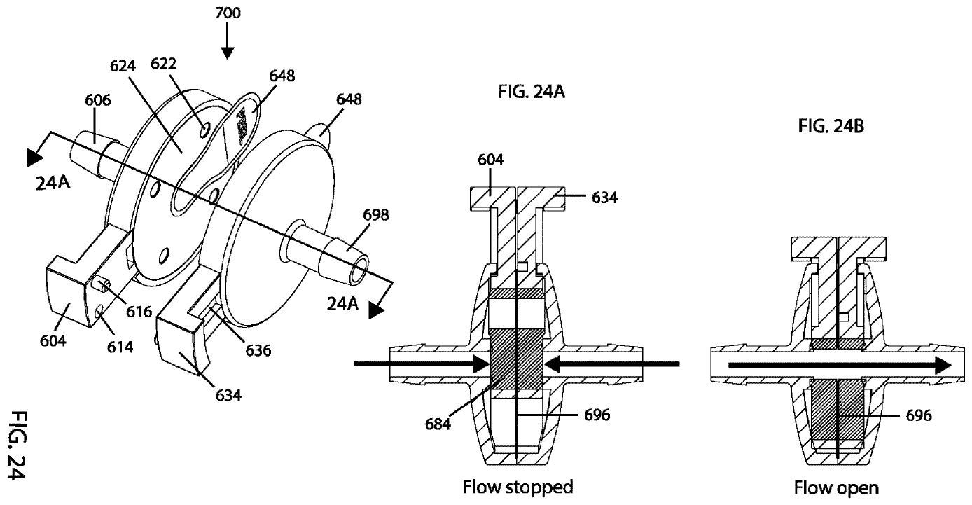

FIG. 22 depicts a perspective view of the genderless expandable valve 600 of FIGS. 19-20 according to some embodiments of the disclosure. The rotary handle 608 is in an open position. FIG. 23 depicts a sectional view of the genderless expandable valve of FIGS. 19-22, according to some embodiments of the disclosure. FIG. 23A shows the genderless expandable valve in a closed position. The sliders 604 and 634 are in an open position, as is the rotary handle 608. As can be seen, the rotary handle 308 is in an open position in FIG. 23B, wherein a flow path can be incoming from inlet port 606 and is directed out through outlet port 628, as both sliders 604 and 634 are in an open position. The silicone area 684 in slider 638, which is in a closed position, provides support so that incoming fluid cannot force off the cap 618. It is possible to take off the cap 618 and provide a port (not shown) instead. The genderless expandable valve 600, though the rotary handle 608 is in a closed position, could then slide the slider 638 into an open position, wherein the flow of liquid would flow straight though the genderless expandable valve 600, for example, forming a flow path for sampling. FIG. 23C shows a front perspective view cross-section of the genderless expandable valve 600 in an open position. Also shown is o-ring 696, which can provide a seal between, for e.g., two adjacent sliders 604, 634. Alternately, or in addition to, an o-ring 696 can be disposed between the slider 638 and the cap 618.

FIG. 24 depicts a partially exploded view of a second genderless expandable valve 700, according to some embodiments of the disclosure. The second genderless expandable valve 700 comprises the inlet port 606 and an outlet port 698. As with the genderless expandable valve 600, described above, the slider 604 comprises a post 616 for engaging a corresponding hole 614 on the slider 634. The second genderless expandable valve 700 further comprises the valve disk 624 having holes 622 and, optionally, peel strip 648. A mirror image slider 634 is shown adjacent to slider 604. Joining the slider 604 with the slider 634 forms the second genderless expandable valve 700, having an o-ring 696 disposed therebetween. As depicted in FIG. 24A, when sliders 604 and 634 are pulled in an outward position, second genderless expandable valve 700 is in a closed position. When each of the sliders 604, 634 are pushed inward, each is in an open position, allowing fluid to flow therethrough, as shown in FIG. 24B, i.e., forming a flow path. Any of the embodiments of the valves of FIGS. 19-24 are also capable of being used in the inlet ports, outlet ports, and vent ports in any of the cassettes and systems described above with respect to other connectors and valves. Any of the embodiments of FIGS. 19-24 may be integrated as sterile connectors and/or barriers within any of the cassettes or stackable devices as described herein.

While various aspects and embodiments have been disclosed herein, other aspects, embodiments, modifications and alterations, will be apparent to those skilled in the art upon reading and understanding the preceding detailed description. The various aspects and embodiments disclosed herein are for purposes of illustration and are not intended to be limiting. It is intended that the present disclosure be construed as including all such aspects, embodiments, modifications and alterations insofar as they come within the scope of the appended claims or the equivalents thereof.

What is claimed is:

1. A rotary genderless valve, comprising:
a body having a body hole;
a body cover for attaching to the body, the body cover comprising a cover hole;
a handle, wherein the handle comprises a closed side and an open side actuated by a rotary motion; and
a rotary slide attached to the handle capable of partial rotation, wherein the rotary slide has an elastomeric slide that further comprises a rotary slide hole;
wherein, the rotary genderless valve is configured to be joined with a second genderless valve having a second rotary slide and a second rotary slide hole, and wherein the rotary slide hole and the second rotary slide hole are in fluid communication with one another.

2. The rotary genderless valve of claim 1, wherein a film is sealed to or releasably attached to the elastomeric slide.

3. The rotary genderless valve of claim 1, wherein the rotary slide is made of a silicone material or a thermoplastic elastomer.

4. The rotary genderless valve of claim 3, wherein the thermoplastic elastomer is a polyolefin, such as polypropylene, polyethylene, ethylene-propylene rubbers, ethylene-propylene diene rubbers, or block copolymers of polyolefins.

5. The rotary genderless valve of claim 1, wherein the body comprises body chamfers adjacent to the body hole.

6. The rotary genderless valve of claim 1, wherein the rotary slide comprises raised areas adjacent the rotary slide hole.

7. The rotary genderless valve of claim 1, wherein the rotary genderless valve is capable of creating an aseptic condition.

8. The rotary genderless valve of claim 1, wherein an air gap exists between the rotary slide and the body cover.

9. The rotary genderless valve of claim 1, further comprising a port in fluid communication with the body hole.

10. The rotary genderless valve of claim 2, wherein the rotary genderless valve and the film are sterilized.

11. (canceled)

12. (canceled)

13. (canceled)

14. (canceled)

15. (canceled)

16. (canceled)

17. (canceled)

18. (canceled)

19. (canceled)

20. (canceled)

21. A genderless valve, comprising:
a body;
a handle, wherein the handle comprises a closed side and an open side;
a port attached to the body;
an elastomeric slide attached to the handle, wherein the elastomeric slide comprises an orifice, wherein the elastomeric slide moves linearly when a force is applied to the handle; and
a film sealed to or releasably attached to the elastomeric slide.

22. The genderless valve of claim 21, wherein the film is attached to the elastomeric slide.

23. The genderless valve of claim 21, wherein the elastomeric slide is made of a silicone material or a thermoplastic elastomer.

24. The genderless valve of claim 21, wherein the film and the genderless valve are sterilized.

25. The genderless valve of claim 21, wherein the genderless valve is capable of creating an aseptic condition.

26. The genderless valve of claim 23, wherein the thermoplastic elastomer is a polyolefin, such as polypropylene, polyethylene, ethylene-propylene rubbers, ethylene-propylene diene rubbers, or block copolymers of polyolefins or silicone(s).

27. A genderless expandable valve, comprising:
an inline valve having a first valve disk, wherein a first hole is within the first valve disk;
a center valve having a second slider and a second valve disk having a second hole, wherein the second slider can slide to be in an open position or a closed position; and
a cap or an outlet port, wherein the inline valve comprises an inlet port and a first slider, wherein the first slider of the inline valve can slide to be in an open position or a closed position, and wherein a flow path is formed through the first valve disk of the inline valve and the second valve disk of the center valve when the inline valve is in the open position and the center valve is in the open position.

28. The genderless expandable valve of claim 27, further comprising a third slider, and a hub having a rotary handle for opening the center valve.

29. The rotary genderless valve of claim 1, wherein the body cover further comprises alignment holes.

30. A filtration device comprising:
a first genderless valve according to claim 21; and
a second genderless valve having a second orifice, wherein the orifice of the genderless valve and the second orifice of the second genderless valve are in fluid communication with one another.
